(19) United States
(12) Patent Application Publication
(10) Pub. No.: US 2012/0046484 A1
(43) Pub. Date: Feb. 23, 2012

(54) Monomers and Macromers for Forming Hydrogels

(75) Inventors: Ramesh Iyer, Harleysville, PA (US); Peter McKenna, Suffolk (GB); Mark Smallridge, Hythe (GB); Melissa Matthews, Hampshire (GB); Anbu Natesh, North Wales, PA (US); Jody Baker, Hampshire (GB)

(73) Assignee: Cognis IP Management GmbH, Dusseldorf (DE)

(21) Appl. No.: 13/256,274

(22) PCT Filed: Mar. 4, 2010
(86) PCT No.: PCT/EP2010/001336

§ 371 Date: Sep. 13, 2011

Related U.S. Application Data
(60) Provisional application No. 61/160,093, filed on Mar. 13, 2009.

(51) Int. Cl. C07F 7/10; C07C 271/22

(52) U.S. Cl. 556/420; 560/160; 560/158

(57) ABSTRACT

Disclosed are new compounds having utility in applications, including as reactants and intermediates in for the formation of polymers and polymeric materials especially useful as hydrogels for ophthalmic lenses.

### FIELD OF THE INVENTION

The invention is related to novel compounds having utility in the formation of polymeric materials and particularly in the formation of hydrogels. The invention also relates to the use of such materials in the field of hydrogel contact lenses, wound healing, controlled drug delivery, medical devices, catheters, stents and tissue engineering.

### BACKGROUND

A hydrogel is a hydratable crosslinked polymeric system. Hydrogels useful in many applications are also oxygen permeable and bio-compatible, making them preferred materials for producing bio-medical devices and in particular contact or intraocular lenses. Conventional hydrogels are prepared from monomeric mixtures predominantly containing hydrophilic monomers such as 2-hydroxyethyl methacrylate (HEMA) or N-vinyl pyrrolidinone (NVP) and hydrophobic monomers or macromers to obtain a polymer having a required hydration capacity and oxygen permeability. The oxygen permeability is commonly associated with polymers found from hydrophobic monomers containing siloxane or fluoro polymer moieties. U.S. Pat. Nos. 4,495,313, 4,889,664 and 5,039,459 disclose the formation of conventional hydrogels. The oxygen permeability of the conventional hydrogel materials are related to water content of the materials and is typically below 20-30 barrers. For contact lenses made of the conventional hydrogel materials, the level of oxygen permeability is suitable for short-term wear of the contact lenses; however, that level of oxygen permeability may be insufficient to maintain a healthy cornea during long-term wear of the contact lenses (e.g., 30 days without removal). Efforts have been made and continue to be made to increase the oxygen permeability and water content or hydration of conventional hydrogels without adversely affecting the physical properties of the hydrogel polymers.

One known way to increase the oxygen permeability of hydrogels is to add silicone-containing monomers or macromers to the hydrogel formulation and/or fluorine-containing monomers or macromers in the formulation to produce the hydrogels. Silicone-containing hydrogels generally have higher oxygen permeabilities than conventional hydrogels. Silicone-containing hydrogels have typically been prepared by polymerizing mixtures containing at least one organic silicone-containing monomer and at least one hydrophilic monomer. Either the silicone-containing or the hydrophilic monomer may function as a crosslinking agent (a crosslinking agent is a monomer having multiple polymerizable functionalities) or a separate crosslinking agent may be employed.

The formation of silicone hydrogels has been disclosed in U.S. Pat. Nos. 4,711,943, 4,954,587, 5,010,141, 5,079,319, 5,115,056, 5,260,000, 5,336,797, 5,358,995, 5,387,632, 5,451,617, 5,486,579, 5,789,461, 5,776,999, 5,760,100, 5,849,811 and WO 96/31792, the contents of which references are incorporated herein by reference.

U.S. Pat. No. 3,808,178 discloses the formation of co-polymers with low molecular weight silicone-containing monomers and various hydrophilic monomers. U.S. Pat. No. 5,034,461 describes silicone-containing hydrogels prepared from various combination of silicone-polyurethane macromers and hydrophilic monomers such as HEMA, vinyl pyrollidone (NVP) and/or dimethylacrylamide (DMA). The addition of methacryloxypropyltris(trimethylsiloxy)silane (TRIS) reduces the modulus of such hydrogels, but in many examples the modulus was still higher than may be required.

U.S. Pat. Nos. 5,358,995 and 5,387,632 describe hydrogels made from various combinations of silicone macromers, TRIS, NVP and DMA. Replacing a substantial portion of the silicone macromer with TRIS reduced the modulus of the resulting hydrogels. The two publications from the same author, “The Role of Bulky Polysiloxane Alkylmethacrylates in Polyurethane-Polysiloxane Hydrogels”, J. Appl. Poly. Sci., Vol. 60, 1193-1198 (1996), and “The Role of Bulky Polysiloxanyl Alkylmethacrylates in Oxygen Permeable Hydrogel Materials”, J. Appl. Poly. Sci., Vol. 56, 317-324 (1995) also describe experimental results indicating that the modulus of hydrogels made from reaction mixtures of silicone-macromers and hydrophilic monomers such as DMA decreases with added TRIS.

The use of methacryloxypropyltris(trimethylsiloxy)silane (TRIS) to make hard contact lenses was described in WO 91/10155 and in JP 61123609.

Notwithstanding some degree of success in connection with prior materials used for the formation of polymeric materials, particularly hydrogels, applicants have come to recognize a continuing need for new compounds, compositions, materials, products and methods having an advantageous but difficult to obtain combination of properties. With respect to hydrogels, applicants have come to appreciate the need for hydrogels that are soft, possess high oxygen permeability, suitable water content and sufficient elasticity.

### SUMMARY

Applicants have developed new compounds, and new compositions, having utility in numerous applications, including as reactants and intermediates in the formation of polymers and polymeric materials. In addition, applicants have developed new polymers based upon (that is, formed at least in part from) one or more of the compounds and/or compositions described herein, and preferably one or more of the novel compounds described herein.

One aspect of the present invention provides compounds according to Formula I:

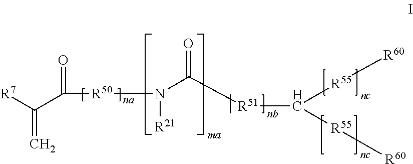

where, provided that the compound has at least two terminal —OH groups or at least one terminal siloxane group, and provided further that if the compound does not have at least two terminal —OH groups, then ma is greater than 0,
where
R⁷ is H or a straight chain or branched, substituted or unsubstituted C1-C4 alkyl group, and in certain preferred embodiments methyl,
each R²¹ is independently H, a C1-C4 alkyl group, or R²³,
   where
   R²³ is R²⁵—O—(CR^{25A}H—CR^{25A}HO)ₓ—CHR^{25A}CR^{25A}H—,
      where
      each R²⁵ is independently a straight chain or branched, substituted or unsubstituted, C1-C4 alkyl group,
      each R^{25A} is independently H, a straight chain or branched, substituted or unsubstituted, C1-C4 alkyl group
      and x is from about 1 to about 50,
each R⁵⁰ is independently a bivalent group selected from R^{50A} and R^{50B}, and each R⁵¹ is independently a bivalent group selected from R^{51A} and R^{51B}
   where
   R^{50A} and R^{51A} are each independently

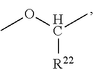

and
R^{50B} and R^{51B} are each independently

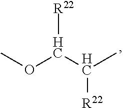

where
each R²² is independently H, a halogen, or a substituted or unsubstituted C1-C4 alkyl, provided preferably that at least one R²² is H,
each R⁵⁵ is independently a bivalent group selected from
   —O—, —NH—, —[CH₂]ₐ—, —[CF₂]_{b}—, —[C(R²²)₂]_{b}—, R^{55A}, R^{55B}, R^{55C}, R^{55D}, R^{55E}, R^{55G}, R^{55H}
   where
   each a is independently from 1 to 10,
   each b is independently from 2 to 50, preferably from 2 to 20 in certain embodiments,
   each R²² is as defined above,
   R^{55A} is

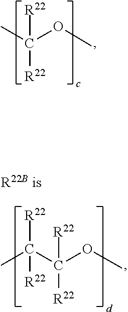

each c is independently 1 to 5,
each d is independently from 8 to 50, and
R^{55C} is

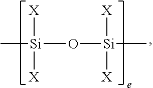

where e is from 1 to 100, more preferably in certain embodiments from 1 to 50, and from 1 to about 30 is certain preferred embodiments,
and

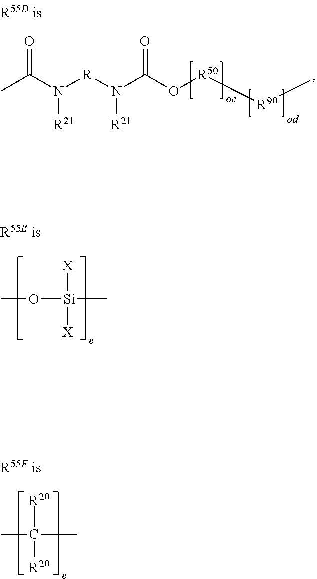

where each R²⁰ is independently H or F,
R^{55G} is

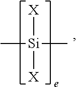

R^{55H} is
—CH₂—R^{55D}—
and
each R⁶⁰ is independently
   H (preferably with not more than one R⁶⁰ being H),
   OH, R²⁶OH, R^{60A}, R^{60B}, R^{60C} and R^{60D},
   where
   R²⁶ is —[CH₂]_{c}— (where c is as defined above)
   R^{60A} is

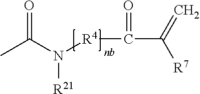

where R⁴ is a C1-C6 alkyl group,
R^{60B} is

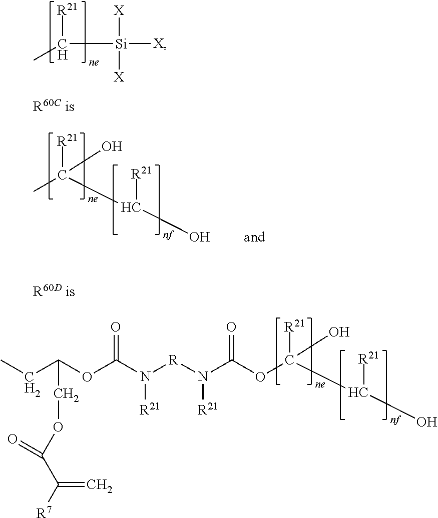

where
each R is independently aryl, cycloalkyl and aliphatic alkyl or aromatic alkyl, polyaromatic, polyaromatic alkyl or polycycloaliphatic alkyl, each X is independently H, an alkyl or haloalkyl moiety having from 1 to about 10 carbon atoms, with and without ether linkages between carbon atoms, or a siloxane group corresponding to —O—Si—R⁹, with each R⁹ being independently a straight chain or branched, substituted or unsubstituted C1-C4 alkyl group, or a phenyl group, and
where
each na, ne and nf is independently from 1 to 4,
ma is 0 or 1,
each nc is independently 0 to 6,
each of nb, ob, oc, and od is independently from 0 to 4.

Another aspect of the invention comprises compositions comprising one or more of the compounds of the present invention in accordance with Formula I. Another aspect of the invention is directed to methods of forming the compounds and compositions of the invention. Another aspect of the invention relates to methods of processing the present compounds and composition, including polymers and polymeric materials formed from there from.

### DETAILED DESCRIPTION OF PREFERRED EMBODIMENTS

### Definitions

For the purposes of convenience, the following definitions apply unless specifically indicated or modified herein in the particular context of use.

The term “C1-C4 alkyl group” means and includes within its scope all alkyl groups having at least one carbon atom but not more than about 4 carbons atoms. Unless specifically indicated herein to the contrary, the term “C1-C4 alkyl group” includes within its scope all straight chain, branched chain, substituted and unsubstituted versions thereof. Similar terminology has the same meaning depending on the numbers used. For example, the term “C1-C6 alkyl group” means and includes within its scope all alkyl groups having at least one carbon atom but not more than about 6 carbons atoms.

The terms “terminal —OH group” and “terminal siloxane-containing group” means a hydroxyl group or a siloxane-containing group bonded to a primary, secondary or tertiary carbon atom.

Unless specifically indicated otherwise herein or clearly indicated otherwise by context, all values for R substituents and subscript values have the same meaning throughout the present specification once defined herein.

### The Compounds

The novel compounds of the present invention, and the new polymers and copolymers, including cross-linked polymers, formed from the present compounds, either alone or in combination one or more of additional monomers and cross-linking agents, are particularly useful in connection with the formation of biomedical materials and products, including catheters and stents, and are especially advantageous in the area of contact lens applications. The new polymers, oligomers, macromers and monomers in accordance with the present invention provide an opportunity to introduce oxygen and in particular hydroxyl groups into the structure of the polymeric material produced there from to increase the hydration capacity of the polymer, and particularly the hydrogel forms thereof, and are especially advantageous in connection with soft contact lens material. The preferred polymers of the present invention have the advantage of having a relatively high polarity while at the same time providing excellent mechanical and physical properties to the contact lens material produced therefrom.

In certain preferred embodiments of the present invention, the compounds of Formula I comprise compounds in which ma is 1, each nc is 0, one R⁶⁰ is OH and the remaining R⁶⁰ is R²⁶OH, which is illustrated below as compounds in accordance with Formula IA:

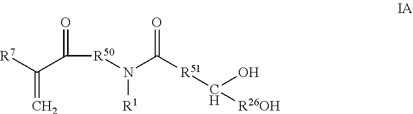

One preferred compound in accordance with this embodiment comprises compounds in which each of na and nb is 1, R⁵⁰ is R^{50B}, R⁵¹ is R^{51A} and R²⁶ is
—CH₂— is illustrated below as Formula IA1:

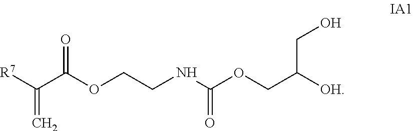

In certain preferred embodiments of the present invention, the compounds of Formula I comprise compounds in which ma is 1, each nc is 0, one R⁶⁰ is H and the remaining R⁶⁰ is a monovalent siloxane-containing group. One preferred compound in accordance with this embodiment is illustrated below as Formula IB:

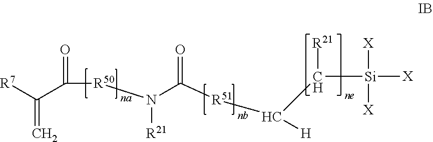

In certain preferred embodiments of Formula IB, each X is —O—Si—R⁹, which is represented by Formula IB1 below:

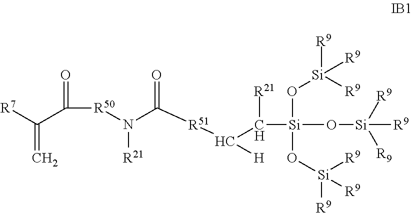

In certain preferred embodiments of Formula IB1, each of na, nb, nc and ne is 1, R⁵⁰ is R^{50B}, R⁵¹ is R^{51A}, and each of R²¹ and R²² is H is illustrated below as Formula IB2:

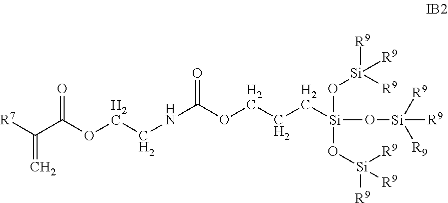

In certain preferred embodiments of the present invention, the compounds of Formula I comprise compounds in which ma is 0, na is 1, with R⁵⁹ being R^{59A}, nb is 0, each nc is 2, with one [R⁵⁵]₂ being —O—R^{55D}— and the other [R⁵⁵]₂ being —R^{55A}—R^{55D}—, with oc and od in each case being 0, in accordance with Formula IC below:

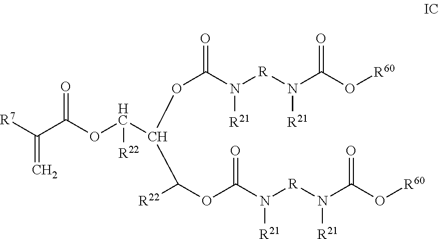

In certain preferred embodiments of the present invention, the compounds of Formula IC comprise compounds in which each R⁶⁰ is R^{60C}, in accordance with Formula IC1 below:

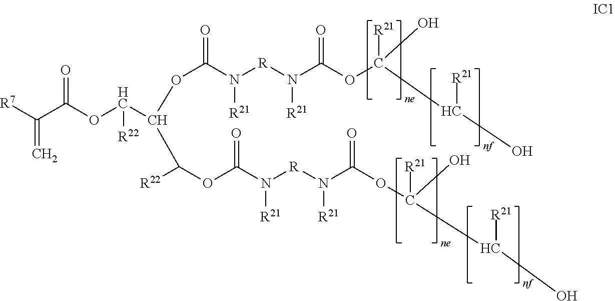

In certain preferred embodiments of the present invention, the compounds of Formula IC1 comprise compounds in which each ne and each of is 1, in accordance with Formula IC2 below:

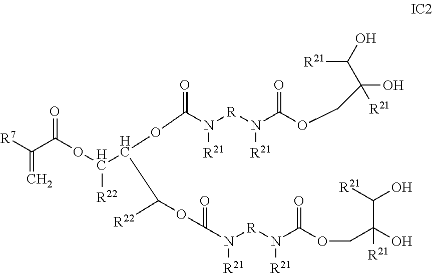

In certain highly preferred embodiments of the compounds in accordance with Formula IC, and more preferably Formula IC1, and even more preferably Formula IC2, each R²¹ and each R²² is H.

In certain preferred embodiments of the present invention, the compounds of Formula I comprise compounds in which ma is 0, na is 1, with R⁵⁰ being R^{50A}, nb is 0, one nc is 2, with one [R⁵⁵]₂ being —O—R^{55D}—, one nc is 5, with [R⁵⁵]₅ being —R^{55A}—R^{55D}—R^{55C}—O—R^{55D}— with oc and od in each case being 0, and one R⁶⁰ being R^{60C} and the other R⁶⁰ being R^{60D} in accordance with Formula ID below:

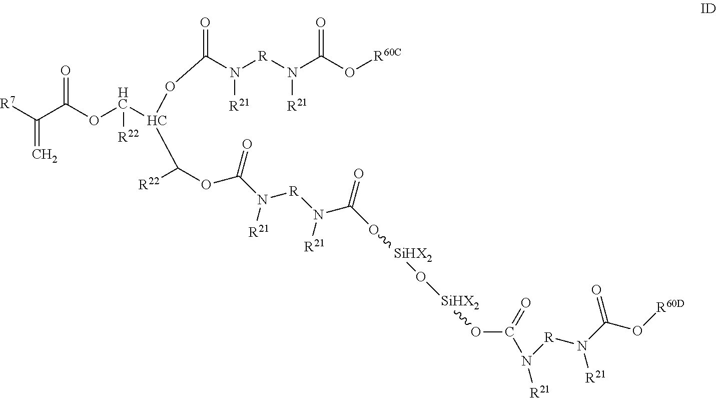

In certain preferred embodiments of the present invention, the compounds of Formula ID comprise compounds in which one R⁶⁰ is R^{60C} and in which the other R⁶⁰ is R^{60D} in accordance with Formula ID1 below:

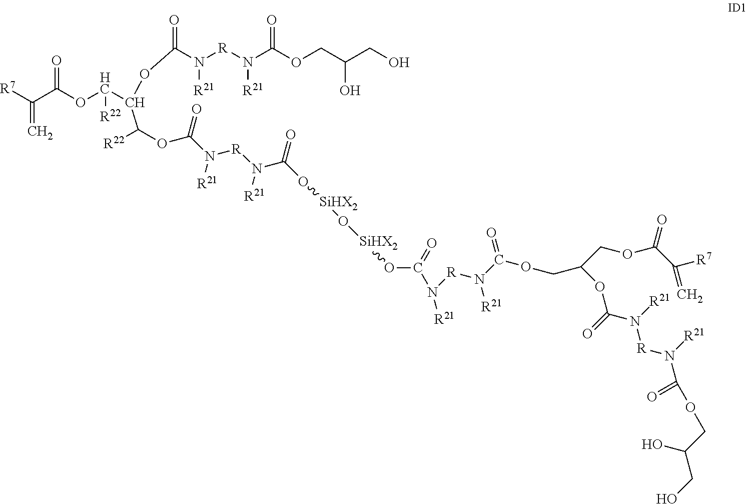

In certain preferred embodiments of the present invention, the compounds of Formula I comprise compounds in which at least one R⁵⁵ is a halogen substituted alkyl, more preferably a fluorine substituted alkyl. In certain of such embodiments, R⁵⁵ is a combination of —[CF₂]_{b}, R^{55A} in which R²² is selected from H and a halogen, preferably F, R^{55B} in which R²² is selected from H and a halogen, preferably F. In certain of such embodiments, particularly those embodiments in which ma is 0, na is 1, with R⁵⁰ being R^{50A}, and nb being 0, then R⁵⁵ comprises such a combination resulting in one or more of the following groups:

—CH₂—O—CF₂—O—(CF₂—CF₂—O)ₓ—(CF₂—O)_{y}—CF₂—CH₂—O—

—CH₂—O—CF₂—O—(CF₂—CF₂—O)ₓ—(CF₂—O)_{y}—CF₂—CH₂—(O—CH₂—CH₂)_{z}—O—

—CH₂—(O—CH₂—CH₂)_{z}—O—CF₂—O—(CF₂—CF₂—O)ₓ—(CF₂—O)_{y}—CF₂—CH₂—(O—CH₂—CH₂)_{z}—O—

where each x, y and z is selected by those skilled in the art to achieve a group average molecular weight ranging from about 1500 to about 4500, more preferably from about 2000 to about 4000. In certain preferred embodiments the average molecular weight of the group is about 2000, and in other embodiments about 2500, and in yet other embodiments about 4000. In certain of such preferred embodiments, the compounds of Formula I comprise compounds in which ma is 0, na is 1, with R⁵⁰ being R^{50A}, nb is 0, one nc is 2, with [R⁵⁵]₂ being —O—R^{55D}—, one nc is 5, with [R⁵⁵]₅ being —R^{55A}—R^{55D}—R^{55AB}—O—R^{55D}— with oc and od in each case being 0, and R55^{AB} being any combination of R^{55A} and R^{55B}, and one R⁶⁰ being R^{60C} and the other R⁶⁰ being R^{60D} in accordance with Formula ID2 below:

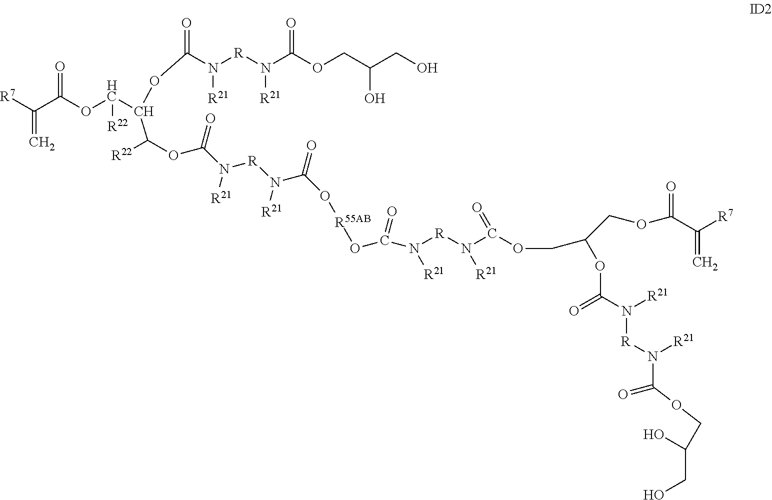

where R^{55AB} is preferably selected from the group consisting of

—CH₂—O—CF₂—O—(CF₂—CF₂—O)ₓ—(CF₂—O)_{y}—CF₂—CH₂—O—

—CH₂—O—CF₂—O—(CF₂—CF₂—O)ₓ—(CF₂—O)_{y}—CF₂—CH₂—(O—CH₂—CH₂)_{z}—O—

—CH₂—(O—CH₂—CH₂)_{z}—O—CF₂—O—(CF₂—CF₂—O)ₓ—(CF₂—O)_{y}—CF₂—CH₂—(O—CH₂—CH₂)_{z}—O—

where subscripts x, y and z are as defined above.

One non-limiting example of the usefulness of the compounds in accordance with Formula I is as monomers, oligomers and macromers which participate in reactions that form a wide variety of useful polymeric materials and products.

One non-limiting example of the usefulness of the compounds in accordance with Formula II is as monomers, oligomers and macromers which participate in reactions that form a wide variety of useful polymeric materials and products, including particularly ophthalmic lens material.

Another aspect of the present invention relates to reactive compositions comprising at least one compound according to Formula I, and to compositions comprising a combination of at least a first compound according to Formula I and at least a second compound, different from said first compound, according to Formula I.

Another aspect of the invention relates to compositions and methods for producing polymeric materials, preferably hydrogel polymers, and even more preferably contact lens material and contact lenses wherein such compositions comprise compounds, and preferably polymeric materials, based on or derived from one or more of the compounds according to Formula I.

One advantage of the compositions and methods of the present invention is that the novel compounds are adaptable to synthesis from relatively inexpensive starting materials and from the use of relatively simple and cost effective procedures. For example, in preferred embodiments certain of the novel compounds in accordance with Formula I are formed utilizing a reactant comprising: a dioxolan compound, and even more preferably a methylated and hydroxylated dioxolan, such as isopropylidene glycerol; and/or isocyanato compound(s); and/or allyloxy alkanediol(s); and/or glycerol ethers, and combinations of any two or more of these.

In preferred embodiments, the compositions and compounds of the invention provide a combination of hydrophilic and hydrophobic groups which, when used to form polymeric materials, and particularly hydrogel compositions, provide a material with a highly advantageous combination of hydrating ability, oxygen transmission properties and mechanical properties.

As those skilled in the art will readily appreciate from the teachings and disclosure contained herein, the novel compounds of the present invention in accordance with Formula I are relatively reactive compounds having substantial and highly desirable utility in a number of applications, including particularly in the formation of polymeric materials. Accordingly, it is contemplated that the reaction products made from the reactive compounds of the present invention will produce, in many embodiments, novel polymeric materials, even in those situations in which the novel reactive compounds of the present invention are reacted with known compounds. Accordingly, one aspect of the present invention relates to novel polymers formed from one or more of the novel reactive compounds of the present invention. As used herein, the term “formed from” encompasses within its meaning any molecule, macromer, oligomer or polymer which is formed, in any substantial part, by a reaction involving any one or more of the compounds of the present invention.

According to certain preferred embodiments, the present invention comprises at least one polymer formed from at least one novel compound in accordance with the present invention.

### The Compositions

One aspect of the present invention is directed to compositions comprising one or more of the compounds of the present invention, and preferably one or more compounds in accordance with Formula I (including all compounds within the general formula and also all those compounds within Formulas IA, IB, IC, ID and IE) and combinations of any two of these. In certain preferred embodiments, the present compositions are reactive compositions comprising at least about 2% by weight, and even more preferably at least about 10% of compounds in accordance with the present invention. In certain preferred embodiments, the compositions of the present invention comprise at least about 50% by weight of compounds in accordance with the present invention, and even more preferably in certain embodiments at least about 40% by weight of compounds in accordance with Formula IC.

In certain preferred embodiments, the compositions include, preferably in an amount of at least about 1%, and more preferably from about 1% to about 90% by weight, more preferably about 1% to about 60%, and even more preferably from about 3% to about 55% by weight of the composition, one or more compounds in accordance with Formula ID, more preferably Formula ID1 or ID2.

As those skilled in the art will appreciate, additional compounds and agents will generally be included in the compositions of the present invention to enhance the reactivity thereof and/or to particularly tailor the compositions for the formation of the desired reaction product or other use, and in particular the desired macromeric, oligmeric and/or polymeric material and even more preferably the desired polymeric material suitable for formation of a hydrogel. It is contemplated that those skilled in the art will be able to readily adapt known comonomers and other agents, such as cross-linking agents, into the compositions of the present invention without undue experimentation in view of the teachings and disclosure contained herein, and all such compositions are within the scope of the present invention.

In particularly preferred embodiments the present compositions include, preferably in an amount of at least about 1%, and more preferably from about 1% to about 80% by weight, and even more preferably from about 40% to about 70% by weight of the composition, one or more co-monomers and/or co-macromers. As used herein, the term co-monomer and co-macromer refers to any compound that is not a novel compound in accordance with the present invention but which acts as a monomer or macromer in the composition. By way of example, but not necessarily by way of limitation, such monomers and/or macromers may include one or more of the following: hydroxyethylmethacrylate, dimethylacrylamide, N-vinylpyrollidone, Methacrylic acid, glycerol monomethacrylate, and combinations of these.

Cross Linking Agents

The compositions can include, preferably in an amount of at least about 0.5%, and more preferably from about 0.5% to about 10% by weight, and even more preferably from about 0.5% to about 5% by weight of the composition, one or more cross linking compounds. By way of example, but not necessarily by way of limitation, such cross-linking agents may include one or more of the following: a compound in accordance with the formula IE and (meth)acrylate crosslinkers such as but not limited to ethyleneglycol dimethacrylate (EGDMA), tri- and/or tetraethyleneglycol dimethacrylate, including all combinations of these two (TEGDMA), butanediol dimethacrylate (BDDMA), (poly)ethyleneglycol dimethacrylate (PEGDMA), and combinations of any two or more of these.

### EXAMPLES

The following examples illustrate the structure and methods of preparation of the compounds of the present invention, as well as the methods of forming such compounds and compositions into polymers and useful articles of the invention. The examples are for illustrative purposes only and are not limiting as to the full scope of the invention. In view of the teachings and disclosures contained herein, including the following examples, it is contemplated that those skilled in the art will be able to readily form the compounds and compositions of the present invention and to utilize such compounds and compositions to form polymeric materials, including particularly hydrogels and articles, such as ophthalmic lenses.

### Example 1

### Synthesis of Compound Corresponding to Formula IA

In a 200 mL three neck round bottomed flask fitted with an air sparge tube, a Teflon stirrer and a gas egress tube connected to an oil bubbler is placed. About 15.5 g of isocyanatoethyl methacrylate, 0.025 g dibutyl tin dilaurate, 100 ppm 2,6-Ditert-butyl-4 methyl phenol and 100 g of dry methyl ethyl ketone are added. The flask is heated under a dry nitrogen sparge to about 55° C. Then using a dry air sparged addition funnel, about 15.84 g of isopropylidene glycerol is added at a rate such that the temperature does not exceed about 65° C. The reaction is complete when there is no NCO peak in an FTIR scan at 2270 cm−1 and the end group analysis of NCO using a dibutylamine titration shows that residual NCO is <0.1%. The resultant mixture is reacted with an MeOH/water mixture to hydrolyze the isopropylidene group. The solvent is removed from the organic layer to obtain monomer product corresponding to Formula IA1 in which R⁷ is methyl.

### Example 2

### Synthesis of Compound Corresponding to Formula IC2

In a 250 mL three neck round bottomed flask fitted with a air sparge tube, Teflon stirrer and a gas egress tube connected to an oil bubbler is placed. About 45.6 g of isophorone diisocyanate, 0.05 g dibutyl tin dilaurate, and 100 ppm of 2,6-ditert-butyl-4-methyl phenol are added and the flask is heated under a dry air sparge to about 55° C. Then using a dry air sparged addition funnel, about 16 g of glycerol monomethacrylate is added at a rate such that the temperature does not exceed about 65° C. The reaction is continued until the % NCO for the adduct is determined by dibutylamine titration to be between 13.8-14.8%. Then about 29.04 g of isopropylidene glycerol is added to the adduct as above and the reaction is continued at about 65° C. until an FTIR scan shows complete absence of an NCO peak at 2270 cm−1 and the end group analysis of NCO using a dibutylamine titration shows that residual NCO is <0.1%. To this mixture about 100 g methyl ethyl ketone, 9 g amberlyst 15 resin and 5 mL water is added and the mixture is stirred at room temperature (about 25° C.) until an FTIR scan shows a complete disappearance of the isopropyl group at 1371 cm−1. The resultant mixture is extracted with an MeOH/water mixture and the solvent is removed from the organic layer to obtain a compound in accordance with Formula IC2 in which each ne and nf is 1 and in which R⁷ is methyl and each R²² and each R²¹ is H.

### Example 3

### Synthesis of Compound Corresponding to Formula IB2

About fifty grams of dry toluene and about 31 g of isocyanatoethyl methacrylate, 0.025 g dibutyl tin dilaurate, 100 ppm 2,6-ditert-butyl-4 methyl phenol are charged to a 250 mL 3-necked RB-flask fitted with a CaCl2 drying tube, condenser and magnetic stirrer. Then about 5.8 g of allyl alcohol is added drop wise at a rate such that any exotherm temperature does not exceed about 75° C. The reaction is continued at about 60° C. until an FTIR scan shows no NCO peak at 2270 cm−1. The dry liquid is then reacted with 29.6 g of tris(trimethylsiloxy)silane in presence of Karstedt catalyst (0.1 molar in xylene, 300 μL) at 65° C., until an FTIR scan showed no SiH peak at 2130 cm−1. The resultant mixture is extracted with an MeOH/water mixture and the solvent is removed from the organic layer to obtain siloxane-containing monomer according to Formula IB2.

### Example 4

### Synthesis of Compound Corresponding to Formula ID1

In a 250 mL three neck round bottomed flask fitted with an air sparge tube, Teflon stirrer and a gas egress tube connected to an oil bubbler is placed 45.6 g of isophorone diisocyanate, 0.05 g dibutyl tin dilaurate, and 100 ppm of 2,6-Ditert-butyl-4 methyl phenol. The flask is heated under a dry air sparge to 55° C. Then using a dry air sparged addition funnel 16 g of Glycerol monomethacrylate is added at a rate such that the temperature does not exceed 65° C. The reaction is continued until the % NCO for the adduct is determined by dibutylamine titration to be between 13.8-14.8%. Then 100 g (0.05 moles) of α,ω.-hydroxypropyl-terminated polydimethylsiloxane KF-6001 from Shin-Etsu (average mol. Wt. of 2000 g/mol) and 100 g of dry toluene are added to adduct as above and the reaction is continued at 65° C. until an FTIR scan shows an absence of a hydroxyl peak at 3400 cm−1 and the end group analysis of NCO using a dibutylamine titration shows that % NCO is between 2.5 and 3.0 for the reaction mixture. Then 13.2 g of isopropylidene glycerol is added to the reaction mixture and the reaction is continued at 65° C. until an FTIR scan shows complete absence of an NCO peak at 2270 cm−1 and the end group analysis of NCO using a dibutylamine titration shows that residual NCO content is <0.1%. To this mixture, 9 g Amberlyst 15 resin and 5 mL water is added and the mixture is stirred at room temperature (25° C.) until an FTIR scan shows a complete disappearance of the isopropyl group at 1371 cm−1. The resultant mixture is extracted with an MeOH/water mixture and the solvent is removed from the organic layer to obtain a compound having the following structure in accordance with Formula ID. Without being bound to any particular theory, it is believed that the compound according Formula ID1 is formed according to the following reaction scheme (with each Y and Z in the following reaction scheme independently corresponding to the substituent defined as X above in connection with Formula I):

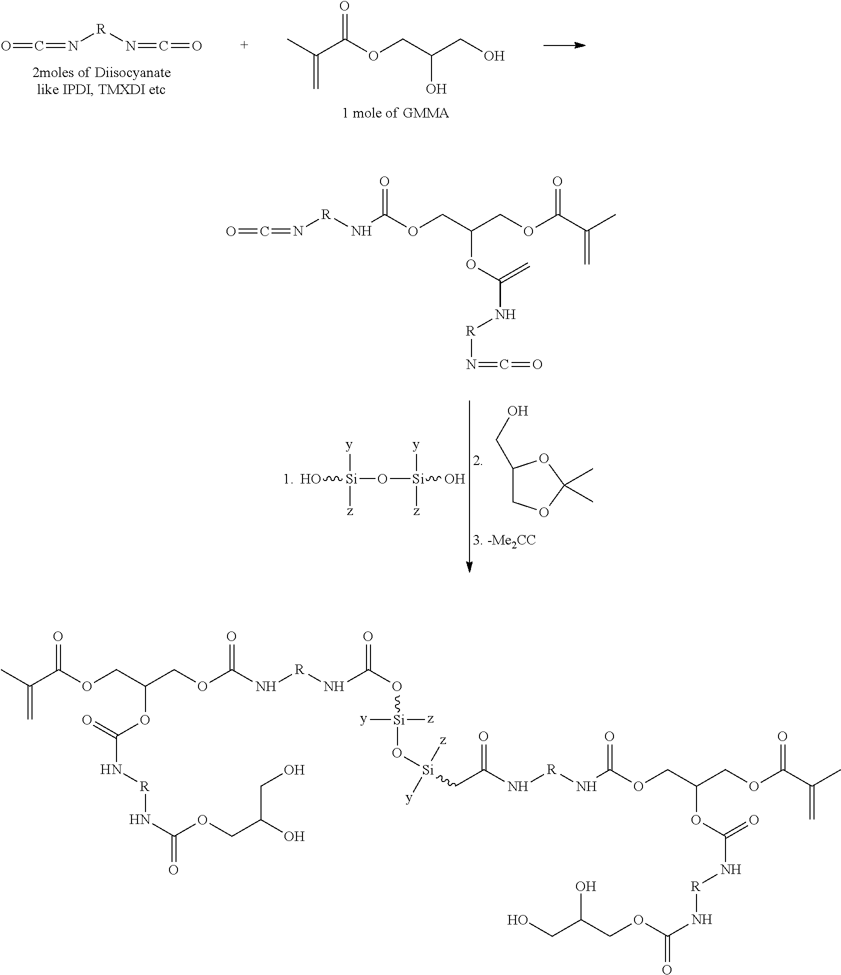

### Example 5

### Synthesis of Compound Corresponding to Formula ID2

In a three neck round bottomed flask fitted with an air sparge tube, stirrer and a gas egress tube connected to an oil bubbler is placed 45.6 g (0.205 mol) of isophorone diisocyanate, 0.1 g Dibutyl tin dilaurate, and 100 ppm of 2,6-Ditert-butyl-4 methyl phenol. The flask is heated under a dry air sparge to 55° C. Then using a dry air sparged addition funnel 16 g (0.10 mol) of glycerol monomethacrylate from example 1 is added at a rate such that the temperature does not exceed 65° C. The reaction is continued until the % NCO for the adducts, as determined by dibutylamine titration, is between 13.8-14.8%. Then 100 g of a dihydroxy functional perfluoropolyether Fomblin® ZDOL 2000 from Solvay Solexis Inc. (Average mol wt about 2000 g/mol) and 100 g of dry toluene is added to the above adduct and the reaction is continued at 65° C. until an FTIR scan shows an absence of hydroxyl peaks 3400 cm−1 and the end group analysis of NCO using a dibutylamine titration shows that residual % NCO is between 2.5 and 3.0 for the reaction mixture. Then 13.2 g of isopropylidene glycerol is added to the reaction mixture and the reaction is continued at 65° C. until an FTIR scan shows complete absence of NCO peak at 2270 cm−1 and the end group analysis of NCO using a dibutylamine titration shows that residual NCO is <0.1%. To this mixture, 9 g Amberlyst 15 resin and 5 mL water is added and the reaction mixture is stirred at room temperature (25° C.) until an FTIR scan shows a disappearance of the isopropyl group at 1371 cm−1. The resultant mixture is extracted with an MeOH/water mixture and the solvent is removed from the organic layer to obtain a fluorinated silicone hydrogel macromer in accordance with Formula ID2.

### Example 6

### Synthesis of Siloxane Crosslinker of Formula I

Fifty gram of dry toluene and 31 g of isocyanatoethyl methacrylate, 0.025 g Dibutyl tin dilaurate, 100 ppm 2,6-Ditert-butyl-4 methyl phenol are charged to a 250 mL 3-necked RB-flask fitted with a CaCl2 drying tube, condenser and magnetic stirrer. Then 13.6 of 1-allyloxy-2,3-propanediol is added drop wise at a rate such that any exotherm temperature does not exceed 75° C. The reaction is continued at 60° C. until an FTIR scan shows no NCO peak at 2270 cm−1. The dry liquid is then reacted with 29.6 g of tris(trimethylsiloxy)silane in presence of Karstedt catalyst (0.1 molar in xylene, 3004) at 65° C., until an FTIR scan showed no SiH peak at 2130 cm−1. The resultant mixture is extracted with an MeOH/water mixture and the solvent is removed from the organic layer to obtain siloxane crosslinker according to Formula I (and identified in Table 1 below as Formula IE1) in which ma is 1, na is 1 with R⁵⁰ being R^{50B}, nb is 1 with R⁵¹ being R^{51A}, with each nc being 1, with one R⁵⁵ being —O— and the other R⁵⁵ being R^{55A} with c being 1, and one R⁶⁰ is R^{60B} with ne being 3 and each X being —O—Si—R7, and the other R⁶⁰ being R^{60A} with nb being 1 and R⁴ being C2 alkyl, and R⁷ and R²¹ being H.

The compositions and compounds of the invention are useful in preparing hydrogels and in particular hydrogels for use in contact lenses, and even more preferably as monomers for preparing hydrogels which require hydrophilic properties for medical devices.

The monomers and macromers of the present invention can be polymerized with known monomers for forming hydrogels to provide hydrogels with unique properties. The following table provides some examples of compositions in accordance with the present invention. The compositions are polymerized using heat or UV and/or visible light or a combination of both using either thermal polymerization initiators such as but not limited to azobisisobutyronitrile or photoinitiators such as but not limited to 2-hydroxy-2-methyl-1-phenyl-propan-1-one, (Tradename Darocur 1173 from Ciba Specialty Chemicals).

TABLE 1
HYDROGEL POLYMER FORMULATIONS

| Product | 1 | 2 | 3 | 4 | 5 | 6 | 7 | 8 | 9 |
|---|---|---|---|---|---|---|---|---|---|
| IA1 | 30 | | | | | | | | 8 |
| IB2 | | 55 | | | | 55 | 55 | 55 | 35 |
| IC2 | | | 20 | | | | | | 10 |
| ID1 | | | | 50 | | | 20 | | 15 |
| ID2 | | | | | 50 | | | 20 | 15 |
| IE1 | | | | | | 3 | 3 | 3 | 3 |
| TEGDMA | 2 | 2 | 2 | | | | | | |
| HEMA | 3.4 | 4.4 | 4.4 | | | | | | |
| TRIS | 50 | 10 | 56 | 30 | 30 | 25 | | | 4 |
| DMA | 14 | 28 | 17 | 19.4 | 19.4 | 16.4 | 21.4 | 21.4 | 9.4 |
| Darocur 1173 | 0.6 | 0.6 | 0.6 | 0.6 | 0.6 | 0.6 | 0.6 | 0.6 | 0.6 |
| Total | 100 | 100 | 100 | 100 | 100 | 100 | 100 | 100 | 100 |

IA1 (Ex 1) Hydrogel forming dihydroxy urethane monmoethacrylate
IB2 (Ex 3) Siloxane functional urethane monomethacrylate
IC2 (Ex 2) Hydrogel forming tetrahydroxy urethane monmoethacrylate
ID1 (Ex 4) Hydrogel forming tetrahydroxy silicone urethane dimethacrylate macromer
ID2 (Ex 5) Hydrogel forming tetrahydroxy flourinated urethane dimethacrylate macromer
IE1 (Ex 6) Siloxane functional urethane dimethacrylate crosslinker
Darocur 1173 2-Hydroxy-2-methyl-1-phenyl-propan-1-one, a photonitiator from Ciba Specialty Chemicals
TEGDMA Triethyleneglycol dimethacrylate
HEMA 2-hydroxyethyl methacrylate
DMA N,N-Dimethylacrylamide

The water content, oxygen permeability and mechanical properties such as elastic modulus, elongation and tensile strength of the hydrogels can be varied over broad ranges by varying the proportions of known hydrogel monomers and macromers. The foregoing examples of formulations for hydrogel polymers can provide hydrogel polymers with required water content, oxygen permeability and physical properties. The combinations of properties make the hydrogels formed from monomers or macromers of the invention useful in many areas of hydrogel utility.

1. A compound according to Formula I: 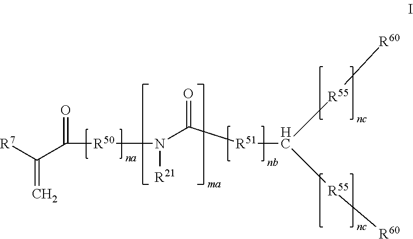
where, provided that the compound has at least two terminal —OH groups or at least one terminal siloxane group, and provided further that if the compound does not have two terminal —OH groups, then ma is greater than 0,
where
R⁷ is H or a straight chain or branched, substituted or unsubstituted C1-C4 alkyl group,
each R²¹ is independently H, a C1-C4 alkyl group, or R²³,
where
R²³ is R²⁵—O—(CR^{25A}H—CR^{25A}HO)ₓ—CHR^{25A}CR^{25A}H—,
where
each R²⁵ is independently a straight chain or branched, substituted or unsubstituted, C1-C4 alkyl group,
each R^{25A} is independently H, a straight chain or branched, substituted or unsubstituted, C1-C4 alkyl group
and x is from about 1 to about 50,
each R⁵⁰ is independently a bivalent group selected from R^{50A} and R^{50B}, and each R⁵¹ is independently a bivalent group selected from R^{51A} and R^{51B}
where
R^{50A} and R^{51A} are each independently 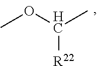
and
R^{50B} and R^{51B} are each independently 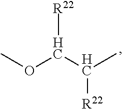
where
each R²² is independently H, a halogen, or a substituted or unsubstituted C1-C4 alkyl, provided that at least one R²² is H,
each R⁵⁵ is independently a bivalent group selected from
—O—, —NH—, —[CH₂]ₐ—, —[CF₂]_{b}—, —[C(R²²)₂]_{b}—, R^{55A}, R^{55B}, R^{55C}, R^{55D}, R^{55E}, R^{55G}, R^{55H}
where
each a is independently from 1 to 10,
each b is independently from 2 to 50,
R^{55A} is 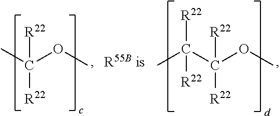
each c is independently 1 to 5,
each d is independently from 8 to 50, and
R^{55C} is 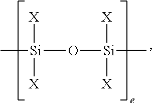
where e is from 1 to 100, 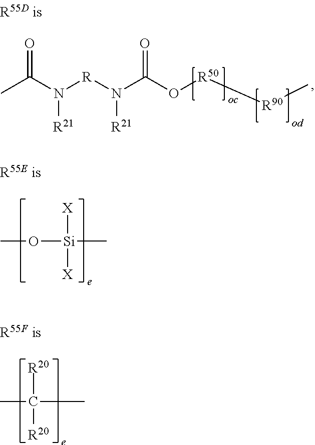
where each R²⁰ is independently H or F, 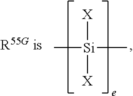
R^{55H} is
—CH₂—R^{55D}—
and
each R⁶⁰ is independently
H, provided that not more than one R⁶⁰ is H,
OH, R²⁶OH, R^{60A}, R^{60B}, R^{60C} and R^{60D},
where
R²⁶ is —[CH₂]_{c}— (where c is as defined above), 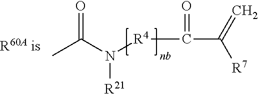
where R⁴ is a C1-C6 alkyl group, 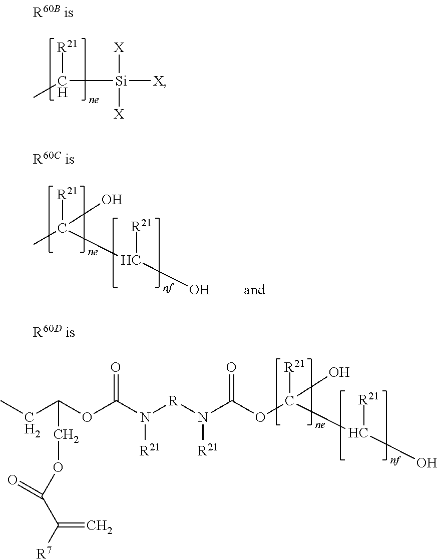
where
each R is independently aryl, cycloalkyl and aliphatic alkyl or aromatic alkyl, polyaromatic, polyaromatic alkyl or polycycloaliphatic alkyl,
each R⁹⁰ is independently bivalent siloxane-containing group, provided that when R⁹⁰ is a bivalent siloxane-containing group, then R⁶⁰ bonded thereto is not H or OH,
each X is independently H, an alkyl or haloalkyl moiety having from 1 to about 10 carbon atoms, with and without ether linkages between carbon atoms, or a siloxane group corresponding to —O—Si—R⁹, with each R⁹ being independently a straight chain or branched, substituted or unsubstituted C1-C4 alkyl group, or a phenyl group and
where
each na, ne and nf is independently from 1 to 4,
ma is 0 or 1,
each nc is independently 0 to 6,
each of nb, ob, oc, and od is independently from 0 to 4.

2. The compound in accordance with claim 1 wherein each R⁷ is H.

3. The compound in accordance with claim 1 wherein at least one R⁷ is H.

4. The compound in accordance with claim 1 wherein at least one R⁷ is a straight chain or branched, substituted or unsubstituted, C1-C4 alkyl group.

5. The compound in accordance with claim 1 wherein b is from 2 to 20.

6. The compound in accordance with claim 1 wherein e is from 1 to 50.

7. The compound in accordance with claim 1 wherein e is from 1 to 30.

8. The compound in accordance with claim 1 wherein ma is 1, each nc is 0, one R⁶⁰ is OH and the remaining R⁶⁰ is R²⁶OH.

9. The compound in accordance with claim 1 wherein at least one of such compounds is in accordance with Formula IA:

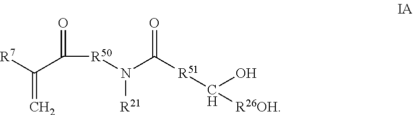

10. The compound in accordance with claim 9 wherein each of na and nb is 1, R⁵⁰ is R^{50B}, R⁵¹ is R^{51A} and R²⁶ is —CH₂—.

11. The compound in accordance with claim 1 wherein at least one of such compounds is in accordance with Formula IA1:

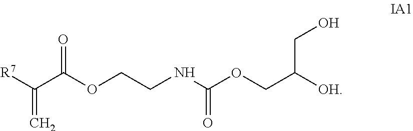

12. The compound in accordance with claim 1 wherein ma is 1, each nc is 0, one R⁶⁰ is H and the remaining R⁶⁰ is a monovalent siloxane-containing group.

13. The compound in accordance with claim 1 wherein at least one of such compounds is in accordance with Formula IB:

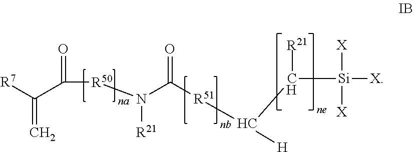

14. The compound in accordance with claim 13 wherein at least one X is —O—Si—R⁹.

15. The compound in accordance with claim 13 wherein each X is —O—Si—R⁹.

16. The compound in accordance with claim 14 wherein at least one of such compounds is in accordance with Formula IB1:

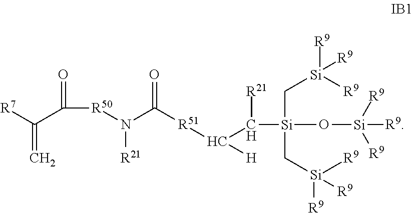

17. The compound in accordance with claim 16 wherein each of na, nb, nc and ne is 1, R⁵⁰ is R^{50B}, R⁵¹ is R^{51A}, and each of R²¹ and R²² is H.

18. The compound in accordance with claim 16 wherein at least one of such compounds is in accordance with Formula IB2:

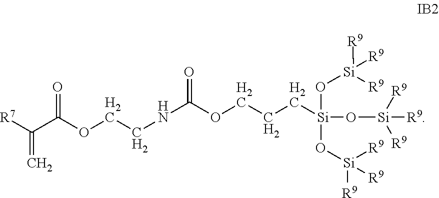

19. The compound in accordance with claim 1 wherein ma is 0, na is 1, with R⁵⁰ being R^{50A}, nb is 0, each nc is 2, with one [R⁵⁵]₂ being —O—R^{55D}— and the other [R⁵⁵]₂ being —R^{55A}—R^{55D}—, with oc and od in each case being 0.

20. The compound in accordance with claim 19 wherein at least one of such compounds is in accordance with Formula IC:

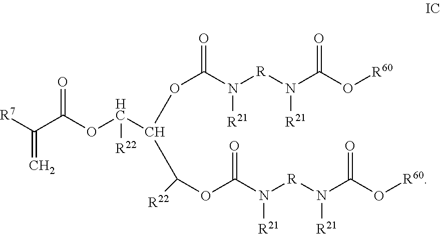

21. The compound in accordance with claim 20 wherein at least one R⁶⁰ is R^{60C}.

22. The compound in accordance with claim 20 wherein each R⁶⁰ is R^{60C} in accordance with Formula IC1 below:

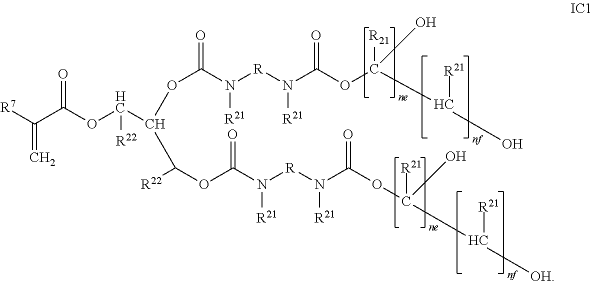

23. The compound in accordance with claim 22 wherein each ne and each of is 1, in accordance with Formula IC2 below:

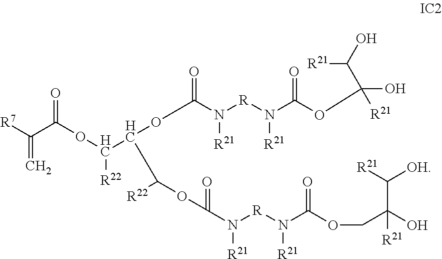

24. The compound in accordance with claim 1 wherein ma is 0, na is 1, with R⁵⁰ being R^{50A}, nb is 0, one nc is 2, with one [R⁵⁵]₂ being —O—R^{55D}—, one nc is 5, with [R⁵⁵]₅ being —R^{55A}—R^{55D}—R^{55C}—O—R^{55D}— with oc and od in each case being 0, and one R⁶⁰ being R^{60C} and the other R⁶⁰ being R^{60D}.

25. The compound in accordance with claim 1 wherein at least one of such compounds is in accordance with Formula ID:

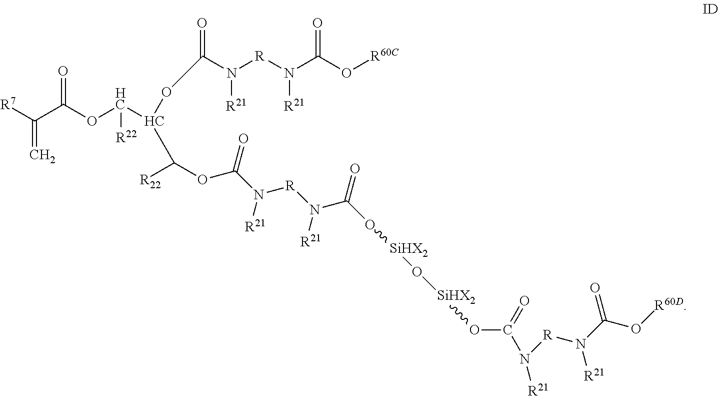

26. The compound in accordance with claim 25 wherein the compound is in accordance with Formula ID1:

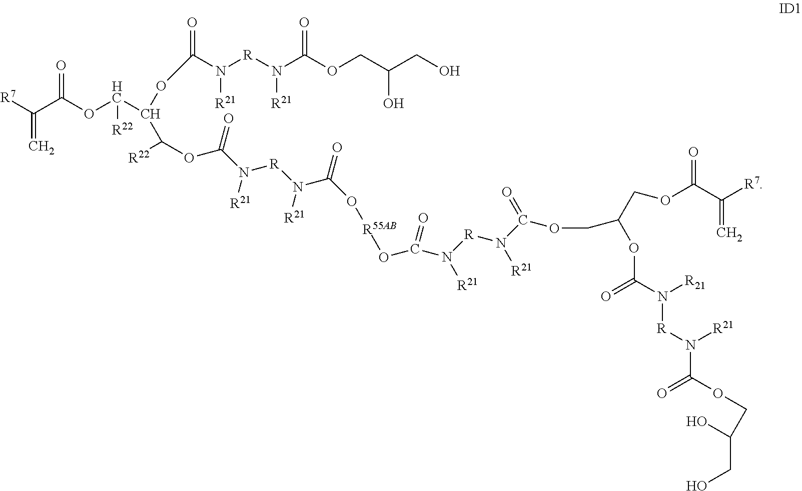

27. The compound in accordance with claim 1 wherein at least one R⁵⁵ is a halogen substituted alkyl.

28. The compound in accordance with claim 1 wherein at least one R⁵⁵ is a fluorine substituted alkyl.

29. The compound in accordance with claim 1 wherein at least one nc is one and at least one R⁵⁵ is —[CF₂]_{b}—, R^{55A} in which R22 is selected from H and a halogen, R^{55B} in which R22 is selected from H and a halogen, and combinations of any two or more of these.

30. The compound in accordance with claim 29 wherein each R22 is selected from H and F.

31. The compound in accordance with claim 1 wherein ma is 0, na is 1, with R⁵⁰ being R^{50A}, nb is 0, and R⁵⁵ comprises a combination resulting in one or more of the following groups:
—CH₂—O—CF₂—O—(CF₂—CF₂—O)ₓ—(CF₂—O)_{y}—CF₂—CH₂—O—
—CH₂—O—CF₂—O—(CF₂—CF₂—O)ₓ—(CF₂—O)_{y}—CF₂—CH₂—(O—CH₂—CH₂)_{z}—O—
—CH₂—(O—CH₂—CH₂)_{z}—O—CF₂—O—(CF₂—CF₂—O)ₓ—(CF₂—O)_{y}—CF₂—CH₂—(O—CH₂—CH₂)_{z}—O—
where each x, y and z is selected to achieve a group average molecular weight ranging from about 1500 to about 4500.

32. The compound in accordance with claim 31 wherein each x, y and z is selected to achieve a group average molecular weight ranging from about 2000 to about 4000.

33. A reactive composition comprising a compound in accordance with claim 1.

34. A monomer in accordance with claim 1.

35. A macromer in accordance with claim 1.

36. An oligomer in accordance with claim 1.

37. A hydrogel formed from a compound in accordance with claim 1.

38. A contact lens formed from a hydrogel in accordance with claim 37.

39. The compound in accordance with claim 1 wherein at least one R⁷ is methyl.
